(19) United States
(12) Patent Application Publication
(10) Pub. No.: US 2008/0003238 A1
(43) Pub. Date: Jan. 3, 2008

(54) Products made with yellow mustard gum

(76) Inventor: Soheil Khajeh Sharafabadi, New Westminster (CA)

Correspondence Address: SOHEIL K. SHARAFABADI, APT 302, 515 ELEVENTH STREET, NEW WESTMINSTER, BC, V3M 4G4, CA

(21) Appl. No.: 11/475,952

(22) Filed: Jun. 28, 2006

(51) Int. Cl. A61K 36/31; A23L 1/222

(52) U.S. Cl. 424/195.18; 424/755; 426/651

(57) ABSTRACT

Yellow mustard gum was used to produce varieties of new foods, cosmetics, paints, and industrial products including but not limited to the following products: nutraceutical beverages, nutraceutical sauces, nutraceutical salad dressings, nutraceutical confectionary products, cosmetics products, pharmaceutical products, paints products and chemical products and were claimed accordingly.

### BACKGROUND AND PRIOR ART

Sharafabdi's 1990 US patent provides the process of extraction of Yellow Mustard Gum with pseudoplastic/shear thinning properties over a wide range of shear rate having excellent emulsifying, suspending, gelling and rheological properties [1]. In this patent application the use of Yellow Mustard Gum in production of new foods, nutraceutical products, cosmetics, pharmaceuticals, paints, chemical products, etc. are invented and described to provide varieties of new useful products.

The following describes a few examples of the numerous patents issued for the use of industrial gums: Reyes (2000) patented an edible gel comprising alginate, pectin or carrageenan [2]. Lewis, et al (2004) patented an improved breakfast cereal biscuit comprising waxy grain [3]. Ehrlich (1987) patented use of pectin in formulation of dry mix food products [4]. The patent issued to Porter et al, (2005) describes varieties of nutritional foods, including the preparation of protein supplemented food products such as beverages, processed meats, frozen desserts, confectionery products, dairy-type products and cereal grain products [5]. And Cassidy (1993) patented use of Carrageenan in production of processed meats for replacement of animal fat [6]. Porter et al, (2004) patented a premix protein supplemented food product [7]. Also Porter et al (2004) obtained a patent for processed meat products including high protein content modified oilseed material [8]. Gryczka et al. (1981) patented an improved process for producing a superior flavor in fermented meat, particularly sausage [9]. Liu (1992) patented a new pharmaceutical composition and processes for treating and preventing diabetes composing of three ingredients of Oleanolic acid, Saponins of Litchi and Kuguasu [10]. Patent issued to Garrity et al, 2004 claimed a nutraceutical beverage by mixing the mangosteen extract with other juices [11]. Bockow (1997) used the following water-soluble thickening agents in manufacturing of antibiotics creams: gum arabic, karaya gum, gum tragacanth, guar gum, carob bean gum, tara gum, pectines, algtnates, carraghenates, agar, furcellaria, starches and the cellulose derivatives, such as methylcellulose, hydroxymethyl cellulose, hydroxyethyl cellulose, hydroxypropyl cellulose, hydroxypropylmethyl cellulose and the synthetic polymers, such as sodium polyacrylate, polyvinyl alcohol, carboxylic polymer derivatives of acrylic acid [12]. The preferred thickening agents used by Dixon, et al (2005) in preparing toothpaste were carboxyvinyl polymers, carrageenan, hydroxyethyl cellulose, laponite and water soluble salts of cellulose ethers such as sodium carboxymethylcellulose and sodium carboxymethyl hydroxyethyl cellulose, and natural gums such as gum karaya, xanthan gum, gum arabic, and gum tragacanth [13]. Schneider, et al, (2005) listed the gel formers, and conditioners customary in use in cosmetics as cellulose derivatives hydroxypropylcellulose, hydroxyethylcellulose, cationically modified celluloses, polysaccharides, e.g. xanthum gum, caprylic/capric triglycerides, sodium acrylates copolymer, polyquaternium-32 (and) paraffinum liquidum (INCl), sodium acrylates copolymer (and) paraffinum liquidum (and) PPG-1 trideceth-6, acrylamidopropyl trimonium chloride/acrylamide copolymer, steareth-10 allyl ether acrylates copolymer, polyquaternium-37 (and) paraffinum liquidum (and) PPG-1 trideceth-6, polyquaternium 37 (and) propylene glycol dicaprate dicaprylate (and) PPG-1 trideceth-6, and polyquaternium [14]. Borchorst 1978 has patented addition of honey as a lubricating agent in shampoo [15], and a Canadian Patent issued to Patel et al. (1972) claimed special effects of use of Benzyl and phenethyl alcohol in shampoo [16]. The binders used by Lant, et al. (2005) in the manufacturing of detergents were Acacia, Alginic Acid, Carbomer, Carboxymethylcellulose sodium, Dextrin, Ethylcellulose, Gelatin, Guar Gum, Hydrogenated vegetable oil type I, Hydroxyethyl cellulose, Hydroxypropyl methylcellulose, Liquid glucose, Magnesium aluminum silicate, Maltodextrin, Methylcellulose, polymethacrylates, povidone, sodium alginate, starch and zein, ethoxylated hexamethylene diamine quaternary compounds, bishexamethylene triamines or other such as pentaamines, ethoxylated polyethylene amines, maleic acrylic polymers [17]. Thickeners, gelling agents, polymers, Polysaccharides, cellulose polymers, natural proteins or synthetic polypeptides were used by Mougin (2004) in manufacturing cosmetics such as lipsticks, foundations, face powders, eyeshadows, eyeliners, and mascaras [18]. Magdina, et al. (2004), used a pre-manufactured thickener E411 FQ In the formulation of latex paints [19]. Useful known thickeners in paint manufacturing described by Wiggins et al. (2003) were casein and alginates, and synthetic materials such as cellulose derivatives, acrylic polymers, and polyurethane polymers [20].

In manufacturing of paints, cosmetics, foods and pharmaceuticals a great number of chemicals have to be blended and mixed to provide a homogeneous uniform substance. In these industrial applications it is desirable to prevent setting and separation of the mixed products. Generally, the chemicals used in cosmetic are classified as: surfactants, foam boosters, conditioning agent, special additives and preservatives, sequestering agent, viscosity modifiers, clarifying agent, fragrances, colors. In paints the chemicals are classified as pigments, extenders, dispersant, biocide, coalescing agent, film formers, and pH controller (Encyclopedia Wikipedia). In foods and drugs, the chemicals are classified as protein fats, carbohydrates, food additives, coloring, and preservatives. The list of chemicals in each category is too long to list. In this application, in the description of the invention and claims these chemicals are incorporated by reference to the list of chemicals found in the encyclopedia of Wikipedia, and the list of chemicals in the FDA available at the http://en.wikipedia.org, and at the http://www.fda.qov.

The use of microbial derived xanthan gum has limitations in drugs, and cosmetics, pharmaceuticals and nutraceutical products. The applicant has discovered and found that yellow mustard gum can provide a solution for providing new products with specific functionality in solving various industrial problems.

The yellow mustard gum in dry form is tasteless and is difficult to consume directly in dry form. The yellow mustard gum contains important nutraceutical compounds providing an extra health benefit to the products made for human consumption. It is therefore desirable to provide a nutraceutical and pharmaceuticals compositions for diabetic patients to quench their thirst as well as providing added nutraceutical benefits of yellow mustard gum for controlling the blood sugars. The applicant has discovered and invented varieties of nutraceutical beverages comprising of yellow mustard gum to provide adequate amount of nutraceutical compounds for enhancing the heath of healthy human as well. Dry mixes of the nutraceutical compositions can also be produced by those skilled in the arts of food and pharmaceutical manufacturing using standard techniques of mixing dry solid fruits and dried juices, sugars, flavors, colors, and other FDA approved food additives in conjunction with dry yellow mustard gum.

A major problem in processed meat products is release of juices from the processed meat products. The applicant has discovered that by addition of yellow mustard gum to meat an excellent emulsion is formed and during the processing of the meat products a strong gel is formed that binds the free water providing a firm strong gel as well as a solid dry meat product without leakage of meat juice which is more appealing to the customers.

Applicant has invented several new processes and novel products incorporating yellow mustard gum. Numerous varieties of nutraceutical compositions, pharmaceuticals compositions, as well as paint and cosmetics products comprising Yellow Mustard Gum are invented, described, produced and claimed taking into account and incorporating by reference the following list of products in the Definition List herein after:

Definition List 1

| Term | Definition |
|---|---|
| Pseudoplastic | A Yellow Mustard Gum having shear |
| Yellow Mustard | thinning properties over a wide range of |
| Gum | sharer rates. |
| Sauce | Any sauces including but not limited to |
| | the followings: oriental soy sauces, |
| | Japanese Teriyaki, Yakitori, Ponzu, |
| | Tsukeyaki Sukiyaki, and Chinese sauces |
| | such as: Szechuan, and tomato sauces, |
| | pizza sauces, barbeque sauces, chili |
| | sauces, and other sauces, etc. |
| Sauce paste | Any sauces paste comprising of yellow |
| | mustard gum as thickening agent and as |
| | a source of nutraceutical compound. And |
| | the said sauce paste will stay on top of |
| | the foods without slip and can be used |
| | as toppings for sausages and hotdogs. |
| Fruit Juices | Any juice derived from fruits or derived |
| | from the fruit juice concentrates such as: |
| | apple juice concentrate, apricot juice |
| | concentrate, banana juice concentrate, |
| | barberry juice concentrates, blueberry |
| | juice concentrate, black berry juice |
| | concentrates, cantaloupe juice |
| | concentrate, cherry juice concentrate, |
| | cranberry juice concentrate, golab |
| | sherbet, grape juice concentrate, |
| | grapefruit juice concentrate, guava juice |
| | concentrates, guava puree, kale juice |
| | concentrate, kiwi fruit juice concentrate, |
| | lemon and lime Juice concentrates, |
| | lychee juice concentrate, mandarins juice |
| | concentrate, mango juice concentrate, |
| | mango puree, mangosteen juice |
| | concentrates, melon juice concentrates, |
| | nashi juice concentrates, nectarines juice |
| | concentrate, orange juice concentrate, |
| | papaya juice concentrate, passion fruit |
| | juice concentrate, peach juice |
| | concentrates, peach puree, pear juice |
| | concentrate, pear puree, parmesan juice |
| | concentrate, parmesan puree, pineapple |
| | juice concentrate, pomegranate juice |
| | concentrate, pomegranate puree, |
| | pomelo juice concentrate, prune juice |
| | concentrate, raspberry juice concentrate, |
| | rose water sherbet, strawberry juice |
| | concentrate, star fruit juice concentrate, |
| | tangerine juice, tamarillo juice |
| | concentrate, , water melon juice |
| | concentrates, etc. |
| Vegetable Juice | Alfalfa juice concentrate, carrot juice |
| | concentrate, celery juice concentrate, |
| | green barley juice concentrate, green |
| | lettuce juice concentrate, parsley juice |
| | concentrate, rhubarb juice concentrate, |
| | spinach juice concentrate, thyme juice |
| | concentrates, and tomato juice |
| | concentrate, etc. |
| Dairy products | Include milks and any products derived |
| | from milk such as and milk shake, ice |
| | creams, sour cream, cheese, sour milk, |
| | yogurt, and yogurt products in |
| | combinations with fruits and |
| | concentrated juices and fruits puree and |
| | jams listed in definition of juices above, |
| | but not limited to the above list. |
| Meat products | Include beef, mutton, pork, lamb, goat, |
| | camel, horse, donkey, dear, turkey, |
| | chicken, pheasant, fish, and meats of |
| | other wild animals. And mixture of |
| | vegetable protein and meats used in |
| | making processed meat products, |
| | canned food sausages, and packaged |
| | foods. |
| Oil | Edible and inedible oils. Edible oils |
| | comprising of animal fats, and vegetable |
| | oil, fats, margarines such as canola oil, |
| | corn oil, sunflower oil, rapeseed oil, |
| | mustard oil, sesame oil, olive oil, cotton |
| | seed oil, and combinations of any two or |
| | more of the said oils known as cooking |
| | oils. Inedible oils mean mineral oils, |
| | petroleum derived oils such as motor oil, |
| | and industrial grades of vegetable oils, |
| | castor oil, used in cosmetics, paints and |
| | machine, and other chemical industries. |
| Colors and Food | Any FDA approved food colors which is |
| coloring: | used in food and pharmaceutical |
| | industries, including but not limited to |
| | the following food colors: yellow, |
| | orange, green, red, purple, blue, and |
| | combinations of any food colors used in |
| | producing desired color of foods and |
| | juices known to those expert in the field. |
| Natural Acids | Any approved FDA natural acids found in |
| | fruits and vegetables including but not |
| | limited to Citric acid, acetic acid, tartaric |
| | acid their soluble and insoluble salts |
| | and combinations of any approved FDA |
| | food colors, and additives used in |
| | producing desired acidity of the foods |
| | and juices as known to those expert in |
| | the field. |
| Cosmetics | Any FDA approved cosmetic products, |
| | toiletry products, cosmetic pastes for |
| | removing dirt and oils from skins, and |
| | skin conditioners including but not |
| | limited to shampoos, soaps, |
| | conditioners, body and face creams, |
| | toothpastes, hand lotions, shaving |
| | creams, perfumes, and makeup, etc. |
| Food products | Any products consumed by human |
| | including, beverages, juices, dairy |
| | products, meat products, salad dressing, |
| | sauces, bread and baking products, |
| | vegetable proteins in combinations with |
| | any products which could be found in |
| | any academic book about foods. |
| Products | Any products made with the yellow |
| | mustard gums wherein, the yellow |
| | mustard gum is used in the products as |
| | emulsifying, thickening, stabilizer, and |
| | viscosity building agent to impart the |
| | desired physical and functional |
| | properties of the products. And also any |
| | products, which contain the |
| | nutraceutical compound of yellow |
| | mustard gum providing the extra health |
| | benefit to human. |
| Musteriyaki | A new sauce, and a new name of a sauce |
| | comprising a mixture of yellow mustard |
| | gum and teriyaki sauce. |
| Industrial Gums | Any gums, mucilage, hydrocolloids, |
| | natural or synthetics which is used in |
| | foods, drugs, cosmetics, paints, and |
| | chemical industries including but not |
| | limited to the following list: gum arabic, |
| | karaya gum, gum tragacanth, guar gum, |
| | carob bean gum, tara gum, pectines, |
| | alginates, carrageenan, agar, furcellaria, |
| | starches, cellulose derivatives, such as |
| | methylcellulose, hydroxymethyl |
| | cellulose, hydroxyethyl cellulose, |
| | hydroxypropyl cellulose, |
| | hydroxypropylmethyl cellulose and |
| | synthetic polymers, such as sodium |
| | polyacrylate, polyvinyl alcohol, |
| | carboxylic polymer derivatives of acrylic |
| | acid, carboxyvinyl polymers, |
| | carrageenan, hydroxyethyl cellulose, |
| | laponite, caprylic/capric triglycerides, |
| | sodium acrylates copolymer, |
| | polyquaternium-32, sodium acrylates |
| | copolymer, acrylamidopropyl trimonium |
| | chloride/acrylamide copolymer, |
| | steareth-10 allyl ether acrylates |
| | copolymer, polyquaternium-37 |
| | polyquaternium 37 (and) propylene |
| | glycol dicaprate dicaprylate (and) PPG-1 |
| | trideceth-6, polyquaternium-7, |
| | polyquaternium-44 , ant other polymers |
| | etc. |

### DESCRIPTION OF THE INVENTION

The object of the invention is to provide new processes for production of numerous new products comprising of yellow mustard gum such as novel nutraceutical compositions, novel dairy products, novel meat products, novel bread and confectionary products, novel pharmaceutical products, novel cosmetic products and novel paint products, and novel chemical products as described and claimed herein after.

A. Nutraceutical Beverage: Applicant has invented varieties of novel nutraceutical beverage compositions, containing yellow mustard gum to provide the added health benefits as described and claimed. In a preferred embodiment of the invention, the beverage comprising of yellow mustard gum is fortified/complemented by the addition of one or more juice concentrates from the following juice concentrates: apple juice concentrate, apricot juice concentrate, banana juice concentrate, barberry juice concentrates, blueberry juice concentrate, black berry juice concentrates, cantaloupe juice concentrate, cherry juice concentrate, cranberry juice concentrate, golab sherbet, grape juice concentrate, grapefruit juice concentrate, guava juice concentrates, guava puree, kale juice concentrate, kiwi fruit juice concentrate, lemon and lime Juice concentrates, lychee juice concentrate, mandarins juice concentrate, mango juice concentrate, mango puree, mangosteen juice concentrates, melon juice concentrates, nashi juice concentrates, nectarines juice concentrate, orange juice concentrate, papaya juice concentrate, passion fruit juice concentrate, peach juice concentrates, peach puree, pear juice concentrate, pear puree, parmesan juice concentrate, parmesan puree, pineapple juice concentrate, pomegranate juice concentrate, pomegranate puree, pomelo juice concentrate, prune juice concentrate, raspberry juice concentrate, rose water sherbet, strawberry juice concentrate, star fruit juice concentrate, tangerine juice concentrate, tamarillo juice concentrate, water melon juice concentrates, etc., and Vegetable juices such as carrot juice concentrate, celery juice concentrate, green barley juice concentrate, green lettuce juice concentrate, parsley juice concentrate, rhubarb juice concentrate, spinach juice concentrate, thyme juice concentrates, and tomato juice concentrate, etc.

For processing of the beverages, in a first step the yellow mustard gum is treated with water. The first step may comprise treating yellow mustard gum in water at a gum concentration of 1% to 35%. In the second step the viscose material from the first step is mixed in a commercial mixer to produce a homogeneous substance. In a third step other ingredients such as citrus juices, ascorbic acids, citric acid, food coloring materials, flavoring agent, and other commonly used substances in juices are added to the resulting viscous material from the second step. In a forth step the resulting viscous liquid from third steps are mixed and homogenized. The resulting homogenized liquid beverage from the forth step is thermally or electro-magnetically processed pasteurized, sterilized and packaged aseptically. As known to the experts in the field the beverage products could be packaged, sealed and sterilized or the beverages could be pasteurized and or sterilized and then packaged aseptically.

In another alternative process and product development, the dried yellow mustard gum was made into ready dry mixes containing sugar, citric acid, ascorbic acid, vitamins, minerals, salt, sweeteners, dry fruits, dry juice concentrates, food coloring, additives, preservatives, etc and packaged in bottles, glasses, jars, cans, pouches etc., as known to those skilled in the art.

B. Diabetic beverages: The applicant has invented numerous beverages to provide the added benefits of the yellow mustard gum for the diabetic patients/individuals. The basic nutraceutical diabetic beverage is a sugar less composition according to formula 3.2.

In another especially convenient version, concentrated diabetic beverages are produced following formula 3.2. In a first step the yellow mustard gum is treated with water. The first step may comprise treating yellow mustard gum in water at a gum concentration of 1% to 35%. In the second step the viscose material from the first step is mixed in a commercial mixer to produce a homogeneous substance. In a third step other ingredients such as, ascorbic acids, citrus juices, citric acid, tartaric acid, food coloring materials, flavoring agent, and other commonly used substances in juices are added to the resulting viscous material from the second step. In a forth step the resulting viscous liquid/paste from third steps are mixed and homogenized. The resulting homogenized paste from the forth step is thermally or electro-magnetically processed and packed aseptically. The diabetic concentrated beverage containing yellow mustard gum were produced containing ascorbic acids, citric acid, food coloring materials, flavoring agent, and other commonly used substances in juices were added to water, mixed homogenized, pasteurized/sterilized and packed (bottles, cans pouches). The concentrations of the ingredients in the Diabetic concentrated beverage were calculated to provide a beverage of formula 3.2 using a supplied standard dispensing spoon and a standard cup for easy preparation of the beverage. Alternatively, the concentrated beverages are packed into a transparent graduated plastic or glass container showing the exact amount required for one serving. Alternatively a small plastic container is packaged which provide the required amount for one serving according to formula 3.2.

Alternatively the Diabetic Dry Mixes were produced to provide a beverage of formula 3.2 A standard dispensing spoon and a standard cup for easy preparation of the beverage are supplied to easily provide the diabetic beverage. Alternatively a small plastic or paper container is packaged to provide the required amount for one serving according to formula 3.2.

The nutraceutical beverage compositions of the present invention can be produced by small and large-scale food processing. In one embodiment of the invented process, the products can be processed through mixing of one or more of the said juice concentrates listed in the definitions above. More preferable embodiments of the beverage form of the invention requires preparation of a slurry of yellow mustard gum added in water at 0.1-2% or (1-10% ) concentrations followed by the addition of the above said juice concentrates followed by mixing in conventional mixers or homogenizers to provide a desired amount of yellow mustard gum in fruit juice. In calculation of the concentration of the, acids, flavor/aroma and preservatives, care must be taken not exceed the allowable limit since concentrated juices contain preservatives as well as acid and sugars. Each concentrated juice product in the market has different composition; therefore blending of products must be carefully done. The manufactured nutraceutical beverage/juice composition should then be pasteurized or sterilized before marketing the products. Varieties of conventional pasteurizing and sterilizing processes can be applied to produce a safe product of the liquid nutraceutical compositions known to those skilled in the art, such as pasteurization, flash pasteurization, batch and continuous sterilization, HTST sterilization, UHT sterilization, pressure sealing, microwave sterilization, UV sterilization, electromagnetic sterilization etc. The liquid nutraceutical compositions could also be produced and marketed in a frozen state, and in a very unconventional method irradiation technique could be applied to sterilize the products as well. Dry powder and premix forms of the nutraceutical compositions can also be produced by those skilled in the arts of food and pharmaceutical manufacturing using standard techniques of mixing dry solid fruits and dried juices, sugars, natural food acids, flavors, colors, and other FDA approved food additives in conjunction with dry yellow mustard gum.

Alternatively, experts in the arts of foods and chemical manufacturing can produce the invented nutaceutical beverage in many forms. In a first step the diabetic beverage concentrate is complemented with the juice concentrates. Providing and manufacturing the liquid beverage and carbonated liquid beverage containing yellow mustard gum and juice concentrates is accomplished by mixing, packing, processing and marketing of the nutraceutical composition. However, other forms of products containing equal amount of yellow mustard gum could be easily produced such as ready mixes/powders containing equal amount of the juice concentrates listed above. Capsule and tablets containing yellow mustard gum as the nutraceutical compositions can be produced and marketed by those skilled in the art through conventional coating materials containing sugars, gelatins, chocolates as well.

C. Varieties of novel food glazes comprising yellow mustard gum were produced to provide new toppings for confectionary products and other food products. In addition to topping and or glazes these nutraceutical glazes provide the extra benefit of the yellow mustard gum. The new functional glazes are prepared with yellow mustard gum in combination with sugars, sweeteners, citric acid, tartaric acid, ascorbic acids, and the salts of said acids, esters, alcohol, table salt, food colorant, food flavors, preservatives, and other naturally chemicals found in fruits, and vegetables listed in the definitions of this application. Alternatively the same glazes without sugar or sweeteners are produced to provide a healthy food for the diabetic patient as well.

D. Novel Sauce Pastes: Ordinary soy sauce and Japanese sauces, which are in liquid state, could not be directly applied on hotdogs and sausages as the liquid sauce slip and run down the foods. The applicant has invented several thick sauce pastes incorporating yellow mustard gum with the liquid commercial sauces available in the market. The new processes were invented for production of thick japanese sauces pastes of Teriyaki, Yakitori, Ponzu, Tsukeyaki Sukiyaki. The invented yellow mustard gum oriental sauce pastes can be applied as toppings on fast foods and other similar foods and snacks, such as hotdogs, sausages, etc. The concentration of the yellow mustard gum in the sauce paste depends on price and economic factors. The more the yellow mustard gum the more is the price of the finished Oriental Sauce Paste products, and the higher its nutraceutical values. It is possible to the experts in the field of food manufacturing and food imitators to produce cheap Oriental Sauce pastes by incorporating yellow mustard gum in combination with other gums such as galactomannan, i. e, guar gum, locust bean gum, agar, starch, CMC, tragacanth, gum Arabic, pectin, fenugreek, gatti, and even xanthan gum. However, this results in lowering the amount of the yellow mustard gum in the final products, therefore, the final cheap products even though is thick and could be used as toppings lack the nutraceutical quality of yellow mustard gum. A few examples of thick oriental sauce paste for use as toppings incorporating other gums are also presented in this application at a gum ratios of (yellow mustard gum: other gums1:9) and (other gums: yellow mustard gum9 1).

E. In another product and process development the nutraceutical yellow mustard gums were used in new product development and manufacturing of nutraceutical dairy products comprising of yellow mustard gum. The products included nutraceutical milk shake, Ice creams, yogurts, sour dairy beverages, sour milk, sour creams, cottage cheese In manufacturing of the new nutraceutical ice creams 100% yellow mustard gum was used as thickening and viscosity builder. Alternatively a mixture of yellow mustard gum and galactomanans at ratio of 1:9 to 9:1 was used in the formulation of ice cream, and ice cream mixes.

F. In another new product and process development the nutraceutical yellow mustard gums were used in new product development and manufacturing of meat and poultry products. Varieties of sausages were produced using poultry (chicken, turkey), pork, and beef products by addition of yellow mustard gum alone or in conjunction with galactomanans. Due to excellent gelling properties of the yellow mustard gum, the process meat products containing yellow mustard gum thus produced showed excellent textures and were almost dry with no sign of water or leakages of meat juice which is a desirable quality in processed meat.

G. In another new product and process development the nutraceutical yellow mustard gum were used in manufacturing of nutraceutical breads, cakes, confectioneries. Bread, cakes and confectioneries were made using grains and cereals, nuts, raisins, spices, oils, salts, sugars, egg whites, favoring, colorings normally used in baking. The new products obtained have excellent properties due to nutraceutical properties of the yellow mustard gum providing extra health benefit to humans.

H. In another new process and product development, the yellow mustard gum was incorporated into pharmaceutical compositions. Diabetic compositions, anti constipation compositions, anti biotic creams & gels, antibacterial creams& gels, anti fungal creams & gels were produced using yellow mustard gum as emulsifying and gelling agent. Antibiotic gels will find applications where it is necessary to apply the gel on the skin without use of oil to keep the skin in dry condition.

I. In another new process and product development, the yellow mustard gum was incorporated into cosmetics products such as shampoos, hair conditioners, toothpastes, mascara, and lipsticks, etc. Due to the excellent emulsifying, suspending and rheological properties of the yellow mustard gum the products obtained were easily stabilized.

J. In another new process and product development, the dried yellow mustard gum was incorporated into paint products. A few new basic paints were produced providing excellent rolling properties and ease of applications owing to the excellent shear thinning properties of yellow mustard gum, which makes it easy to be applied by rollers. The high viscosity of the yellow mustard gum at low or zero shear rates prevent slipping of the paint, and the paint stays on the wall. The low viscosity of mustard gum at high shear rates makes it easy to be applied on the wall and surfaces with little force. This is very important in paint applications, as the workers hand will not be tired due to low force needed to apply the new paint. Due to the excellent suspending properties of the yellow mustard gum the new paints had excellent shelf life and do not settle fast.

Processes and Product Development

### EXAMPLE 1

### Process for Production of the Thick Oriental Sauce Pastes, and other Thick Sauces

In this method various amount of yellow mustard gum ranging from 0.1% to 20% is added to the natural commercial soy sauce or to the prepared Japanese sauces such as Teriyaki, Yakitori, Ponzu, Tsukeyaki Sukiyaki, etc. After a sufficient soaking time the thick sauce was homogenized in industrial homogenizers, and the final products packaged, and sterilized and or pasteurized. The following examples were used to produce various nutraceutical sauce pastes:

### EXAMPLE 1.1

### Soy Sauce Paste

0.1-20% yellow mustard gum, soy sauce.

### EXAMPLE 1.2

### Musteriyaki Sauce Paste

0.1-20 % yellow mustard gum, Shoyu , Champagne, rice vinegar, garlic, cane sugar, olive oil, honey, spices.

### EXAMPLE 1.3

### Musteryaki Sauce Paste

1-20% yellow mustard gum, Champagne, teriyaki sauce.

### EXAMPLE 1.4

### Teriyaki Sauce Paste

0.1-20 % yellow mustard gum, soy sauce, sake, garlic, brown sugar, honey, spices.

### EXAMPLE 1.5

### Teriyaki Paste Sauce

1-20% yellow mustard gum, commercial teriyaki sauce.

### EXAMPLE 1.6

### Punzu Sauce Paste

0.1-20% yellow mustard gum, soy sauce, sake, rice vinegar, garlic, cane sugar, sesame oil, honey, spices, lime Juice.

### EXAMPLE 1.7

### Punzu Sauce Paste

0.1-20% yellow mustard gum, commercial Punzu sauce.

### EXAMPLE 1.8

### Yakitori Sauce Paste

0.1-20% yellow mustard gum, soy sauce, sake, garlic, garlic, cane sugar, honey, ginger.

### EXAMPLE 1.9

### Yakitori Sauce Paste

0.1-20% yellow mustard gum, commercial Yakitori sauce.

### EXAMPLE 1.10

### Tsukeyake Sauce Paste

0.1-20% yellow mustard gum, soy sauce, sake, rice vinegar, cane sugar, honey, spices, ginger, lemon juice.

### EXAMPLE 1.11

### Tsukeyake Sauce Paste

0.1-20% yellow mustard gum, commercial Tsukeyake sauce.

### EXAMPLE 1.12

### Szechuan Sauce Paste

0.1-20% yellow mustard gum, Shoyu, sake, rice vinegar, garlic, cane sugar, sesame oil, chili, ginger.

### EXAMPLE 1.13

### Szechuan Sauce Paste

0.1-20% yellow mustard gum, commercial Szechuan sauce paste.

### EXAMPLE 1.14

### Chili Sauce Paste

0.1 -20% yellow mustard gum, fermented chili, vinegar or acetic acid, spices, salts, preservatives.

### EXAMPLE 1.15

### Chili Sauce Paste

1-20% yellow mustard gum, commercial chili sauce, vinegar or acetic acid, spices, salts, preservatives.

### EXAMPLE 1.16

### Mango Sauce Paste

1-20% yellow mustard gum, fermented mango, vinegar or acetic acid, spices, salts, preservatives.

### EXAMPLE 1.17

### Tomato Sauce Paste

1-20% yellow mustard gum, tomato paste (concentrated tomato juice, dried tomato), vinegar or acetic acid, spices, salts, preservatives.

### EXAMPLE 1.18

### Tomato Sauce Paste

1-20% yellow mustard gum, concentrated tomato juice.

### EXAMPLE 1.19

### Tomato Sauce Paste

1-20% yellow mustard gum, tomato juice, vinegar or acetic acid, spices, salts, preservatives.

### EXAMPLE 1.20

### Tomato Ketchup Paste

1-20% yellow mustard gum, sugar or sweetener, tomato juice, vinegar or acetic acid, spices, salts, preservatives.

### EXAMPLE 1.21

### Pizza Sauce Paste

1-20% yellow mustard gum, tomato juice, vinegar or acetic acid, spices, salts, preservatives.

### EXAMPLE 1.22

### Spaghetti Sauce

1-20% yellow mustard gum, tomato juice, vinegar or acetic acid, spices, salts, preservatives.

### EXAMPLE 2

### Process for Production of the Yellow Mustard Gum Beverages, Concentrated Yellow Mustard Gum Beverages and Yellow Mustard Gum Dry Ready Mixes

### EXAMPLE 2.1

### Yellow Mustard Gum lemon Beverage (No Sugar)

The following formulation used to produces a fruit juice beverage (no sugar) with fresh lemon peals. Ingredients: Yellow Mustard Gum 0.1-1%, Fresh lemon peals 0.1-0.05%, Vitamin C 0.2-0.05%, Citric acid 0.02-0.04%, Sodium citrate 0.02-0.04%, Sodium chloride 0.001-0.005%, potassium sorbate 0.025%, water add up to 100.

### EXAMPLE 2.2

### Yellow Mustard Gum Orange Beverage

Ingredients: Yellow Mustard Gum 0.1-1%, Orange juice concentrate 5.0-6.0%, sugar 8-10%, Vitamin C 0.2-0.05%, Citric acid 0.02-0.04%, Sodium citrate 0.02-0.04%, Sodium chloride 0.001-0.005%, total potassium sorbate 0.025%, total sodium benzoate 0.025%, orange flavor/aroma 0.05-0.1%, water add up to 100.

### EXAMPLE 2.3

### Yellow Mustard Gum Apple Beverage

Ingredients: Yellow Mustard Gum 0.1-1%, Apple juice concentrate 5.0-6.0%, Vitamin C 0.2-0.05%, Citric acid 0.02-0.04%, Sodium citrate 0.02-0.04%, Sodium chloride 0.001-0.005%, total potassium sorbate 0.025%, total sodium benzoate 0.025%, apple flavor/aroma 0.05-01%, water add up to 100.

### EXAMPLE 2.4

### Yellow Mustard Gum Apricot Beverage

Ingredients: Yellow Mustard Gum 0.1-1%, apricot juice concentrate 5.0-6.0%, Vitamin C 0.2-0.05%, Citric acid 0.02-0.04%, Sodium citrate 0.02-0.04%, Sodium chloride 0.001-0.005%, total potassium sorbate 0.025%, total sodium benzoate 0.025%, apricot flavor/aroma 0.05-0.1%, water add up to 100.

### EXAMPLE 2.5

### Yellow Mustard Gum Cherry Beverage

Ingredients: Yellow Mustard Gum 0.1-1%, Cherry juice concentrate 5.0-6.0%, Vitamin C 0.2-0.05%, Citric acid 0.02-0.04%, Sodium citrate 0.02-0.04%, Sodium chloride 0.001-0.005%, total potassium sorbate 0.025%, total sodium benzoate 0.025%, color, cherry flavor/aroma 0.05-0.1%, water add up to 100.

### EXAMPLE 2.6

### Yellow Mustard Gum Banana Beverage

Ingredients: Yellow Mustard Gum 0.1-1%, banana juice concentrate 5.0-6.0%, Vitamin C 0.2-0.05%, Citric acid 0.02-0.04%, Sodium citrate 0.02-0.04%, Sodium chloride 0.001-0.005%, total potassium sorbate 0.025%, total sodium benzoate 0.025%, color, cherry flavor/aroma 0.05-0.1%, water add up to 100.

### EXAMPLE 2.7

### Yellow Mustard Gum Cranberry Beverage

Ingredients: Yellow Mustard Gum 0.1-1%, cranberry juice concentrate 5.0-6.0% Vitamin C 0.2-0.05%, Citric acid 0.02-0.04%, Sodium citrate 0.02-0.04%, Sodium chloride 0.001-0.005%, total potassium sorbate 0.025%, total sodium benzoate 0.025%, color, cherry flavor/aroma 0.05-0.1%, water add up to 100.

### EXAMPLE 2.8

### Yellow Mustard Gum Grape Beverage

Ingredients: Yellow Mustard Gum 0.1-1%, grape juice concentrate 5.0-6.0%, Vitamin C 0.2-0.05%, Citric acid 0.02-0.04%, Sodium citrate 0.02-0.04%, Sodium chloride 0.001-0.005%, total potassium sorbate 0.025%, total sodium benzoate 0.025%, color, cherry flavor/aroma 0.05-0.1%, water add up to 100.

### EXAMPLE 2.9

### Yellow Mustard Gum Raspberry Beverage

Ingredients: Yellow Mustard Gum 0.1-1%, raspberry juice concentrate 5.0-6.0% Vitamin C 0.2-0.05%, Citric acid 0.02-0.04%, Sodium citrate 0.02-0.04%, Sodium chloride 0.001-0.005%, total potassium sorbate 0.025%, total sodium benzoate 0.025%, color, cherry flavor/aroma 0.05-0.1%, water add up to 100.

All other beverages are produced according to the above procedures.

### EXAMPLE 2.10

### Yellow Mustard Gum Dry Ready Mix

A dry mix comprising of dry yellow mustard gum, ascorbic acids, citric acid, sodium citrate, sugars, sweeteners, potassium sorbate, sodium benzoate, sodium chloride and preservatives color, aroma flavor are mixed, packaged in bottles, glass, cans, pouches.

### EXAMPLE 3

### Diabetic Beverage

### EXAMPLE 3.1

### Diabetic Lemon Beverage

Yellow Mustard Gum 0.1-1%, Fresh lemon peals 0.1-0.05%, Vitamin C 0.2-0.5%, Citric acid 0.02-0.04%, Sodium citrate 0.02-0.04%, Sodium chloride 0.001-0.005%, potassium sorbate 0.025%, total sodium benzoate 0.025%, color, flavor/aroma 0.05-0.1% , water add up to 100.

### EXAMPLE 3.2

### Diabetic Lemon Beverage

Yellow Mustard Gum 0.1-1%, lemon juice concentrate 3-5%, Vitamin C 0.2-0.5%, Citric acid 0.02-0.04%, Sodium citrate 0.02-0.04%, Sodium chloride 0.001-0.005%, potassium sorbate 0.025%, total sodium benzoate 0.025%, color, flavor/aroma 0.05-0.1% , water add up to 100.

### EXAMPLE 3.3

### Diabetic Concentrated Beverage

Yellow Mustard Gum 10%,Vitamin C 0.2-0.5%, Citric acid 0.02-0.04%, Sodium citrate 0.02-0.04%, Sodium chloride 0.001-0.005%, potassium sorbate 0.025%, sodium benzoate 0.025%, flavor, color, color, flavor/aroma 0.05-0.1% , water add up to 100.

### EXAMPLE 3.4

### Diabetic Dry Mix

The ready mix containing dry yellow mustard gum were produced containing ascorbic acids, citric acid, sodium citrate, sodium chloride, potassium sorbate, food coloring materials, flavoring agent, and other commonly substances found in juices were mixed and packed (bottles, cans, pouches, tablets, capsules).

### EXAMPLE 4

### Food Glazes

The following general formula was used to prepare various food glazes, By substitution of different flavors, and colors varieties of glazes could be produced without departing from the scope of the invention by incorporation of Yellow Mustard Gum as the main source of thickening and jelling agent.

### EXAMPLE 4.1

### Yellow Mustard Gum

0.1-1%, Vitamin C 0.2-0.5%, Citric acid 0.02-0.04%, Sodium citrate 0.02-0.04%, Sodium chloride 0.001-0.005%, sugars, sweeteners 6-15%, potassium sorbate 0.025%, flavour, color, water add upto 100.

### EXAMPLE 4.2

Yellow Mustard Gum galactomanan with ratio of (1-9 to 9-1) 0.1-1%, Vitamin C 0.2-0.5%, Citric acid 0.02-0.04%, Sodium citrate 0.02-0.04%, Sodium chloride 0.001-0.005%, sugars, sweeteners 6-15%, potassium sorbate 0.025%, aroma or flavor, color, water add up to 100.

### EXAMPLE 5

### Dairy Products

Several diary products containing yellow mustard gum were made. Varieties of dairy products were made using yellow mustard gum as the thickener and viscosity buildup agent, the following four formulations are provided as reference however, the formulations and products are not limited to the following as could be understood by those skill in the arts several variations within any given formula is possible using the yellow mustard gum:

### EXAMPLE 5.1

### Milk Shake Plain

Add 0.1-0.5% yellow mustard gum to the milk, homogenize pasteurize and pack.

### EXAMPLE 5.2

### Milk Shake Coffee

Yellow mustard gum 0.1-0.5%, sugar 2-5%, 0.1-0.5% Coffee, add up with milk, homogenize, pasteurize, and pack.

### EXAMPLE 5.3

### Milk Shake Chocolate

Yellow mustard gum 0.1-0.5%, sugar 2-5%, chocolate 0.1-1%, add up with milk, homogenize, pasteurize, and pack.

### EXAMPLE 5.4

### Milk Shake Vanilla

Yellow mustard gum 0.1-0.5%, sugar 2-5%, vanilla 0.1-0.05%, add up with milk, homogenize, pasteurize, and pack.

### EXAMPLE 5.5

### Milk Shake Mix

Yellow mustard gum and galactomanan (ratio ranging from 0.1-0.04 to 0.4-0.1%), sugar 2-5%, Cocoa 0.5-1%, 0.5% Coffee, add up with milk, homogenize, pasteurize, and pack.

### EXAMPLE 5.6

### Milk Shake Chocolate

Yellow mustard gum and galactomanan ratio ranging from 0.1-0.04 to 0.4-0.1% , sugar 2-5%, chocolate 0.1-1%, add up with milk, homogenize, pasteurize, and pack.

### EXAMPLE 5.8

### Milk Shake Chocolate

Yellow mustard gum and galactomanan ratio ranging from 0.1-0.04 to 0.4-0.1%, chocolate 0.1-1%, add up with milk, homogenize, pasteurize, and pack (no sugar).

### EXAMPLE 5.9

### Milk Shake Vanilla

Yellow mustard gum 0.1-0.5%, vanilla 0.1-0.05%, add up with milk, homogenize, pasteurize, and pack (no sugar).

### EXAMPLE 5.10

### Ice Cream Mix

Yellow mustard gum, milk powder, sugar, acids, salts, whey powder, galactomanan, vegetable protein, lecithin, food coloring, flavors, dry fruit chunks, nuts, etc.

### EXAMPLE 5.11

### Ice Cream

Milk powder or milk, yellow mustard gum, sugar, acids, salts, whey powder, vegetable protein, with or without lecithin, food coloring, dry fruit chunks, pistachios, nuts, flavors, water, etc.

### EXAMPLE 5.12

### Ice Cream

Milk powder or milk, yellow mustard gum, galactomanan, sugar, acids, salts, whey powder, vegetable protein, with or without lecithin, food coloring, dry fruit chunks, pistachios, nuts, flavors, water, etc.

### EXAMPLE 6

### Salad Dressing

### EXAMPLE 6.1

### A Low Calorie No Fat Salad Dressing

A composition containing yellow mustard gum, vinegar, citric acid or lemon juice, salt, sugar, spices, herbs, garlic, olive, water to give a 6-14 kilocalories per table spoon.

### EXAMPLE 6.2

### A Low Calorie No Fat Salad Dressing

A composition containing, yellow mustard gum galactomanan, vinegar, citric acid or lemon juice, salt, sugar, spices, herbs, garlic, olive, water to give a 6-14 kilocalories per table spoon, mustard seed, mustard bran.

### EXAMPLE 6.3

### Dry Mix Salad Dressing

A dry composition containing yellow mustard gum, citric acid, salt, sugar, spices, herbs, garlic, onion, mustard seed, mustard bran.

### EXAMPLE 6.4

### Dry Mix Salad Dressing

A dry composition containing yellow mustard gum, galactomanan, citric acid, salt, sugar, spices, herbs, garlic, onion, mustard seed, mustard bran.

### EXAMPLE 6.5

### Mayonnaise

A composition containing: yellow mustard gum, vegetable oil, salt, sugar, garlic, onion spices, dry vegetable, and mustard protein.

### EXAMPLE 6.6

### Mayonnaise with Egg Yolk

Composition containing: yellow mustard gum, vegetable oil, salt, sugar, garlic, vinegar, spices, mustard protein, and egg yolk.

### EXAMPLE 6.7

### Mayonnaise with White Egg

Composition containing: yellow mustard gum, vegetable oil, salt, sugar, vinegar, garlic, spices, mustard protein, and egg yolk.

### EXAMPLE 6.8

### Mayonnaise with White Whole Eggs

Composition containing: yellow mustard gum, vegetable oil, salt, sugar, garlic, vinegar, spices, mustard protein, and whole egg.

### EXAMPLE 6.9

### Honey Salad Dressing

Yellow mustard gum 1%, honey 8%, lemon juice 30%, ground pepper, 0.5%, garlic 0.5%, olive oil 60%. Dissolve yellow mustard gum in lemon juice, add honey, pepper, garlic and stir, then add olive oil homogenize in blender.

### EXAMPLE 7

### Processed Meat Products

Several process and product were developed using the nutraceutical yellow mustard gums in manufacturing new meat product. Varieties of sausages were produced using poultry (chicken, turkey), pork, and beef products by addition of yellow mustard gum in conjunction with galactomanans. Due to excellent gelling properties of the yellow mustard gum, the yellow mustard gum processed meat products has excellent textures and were almost dry with no sign of water or meat juice leakages, which is a desirable quality in processed meat. Sufficient amounts of ground meat, fats vegetable protein, spices, yellow mustard gum, salts, sugar, preservatives were mixed thoroughly, and made Into an emulsion then packaged in the natural casing, and then processed.

### EXAMPLE 7.1

### Beef Product

Yellow mustard gum 0.5-2%, ground or minced beef 10-40%, vegetable protein or soy protein 2-10%, pea powder 1-5%, salt HCl 0-5%, sodium nitrite 0.1-1.9%, phosphate 0-5%, sodium ascorbate 0.04-0.07 , glucose 0.03-0.6, spices, water 20-50%.

### EXAMPLE 7.2

### Pork Product

Yellow mustard gum 0.5-2%, ground or minced porkl 0-40%, vegetable protein or soy protein 2-10%, pea powder 1-10%, 0-5% salt HCL, sodium nitrite 0.1-1.9%, phosphate 0-5%, sodium ascorbate 0.04-0.07 , glucose 0.03-0.6, spices, water 20-50%.

### EXAMPLE 7.3

### Mixed Pork and Beef Product

Yellow mustard gum 0.5-2%, ground or minced pork meat 10-40%,ground or minced beef meat 5-20, vegetable protein or soy protein 2-10%, pea powder 1-10%, 0-5% salt HCl, sodium nitrite 0.1-1.9%, phosphate 0-5%, sodium ascorbate 0.04-0.07 , glucose 0.03-0.6, spices, water 20-50%.

### EXAMPLE 7.4

### Chicken Product

Yellow mustard gum 0.5-2%, %, ground or minced chicken 5-20, vegetable protein or soy protein 2-10%, pea powder 1-10%, 0-5% salt HCl, sodium nitrite 0.1-1.9%, phosphate 0-5%, sodium ascorbate 0.04-0.07 , glucose 0.03-0.6 spices, water 20-50%.

### EXAMPLE 7.5

### Turkey Product

Yellow mustard gum 0.5-2%, %, ground or minced turkey 5-20, vegetable protein or soy protein 2-10%, pea powder 1-10%, 0-5% salt HCl, sodium nitrite 0.1-1.9%, phosphate 0-5%, sodium ascorbate 0.04-0.07, glucose 0.03-0.6 spices, water 20-50%.

### EXAMPLE 7.6

### Mixed Chicken and Turkey Product

Yellow mustard gum 0.5-2%, ground or minced chicken 5-20%, ground or minced turkey 5-20, vegetable protein or soy protein 2-10%, pea powder 1-10%, 0-5% salt HCl, sodium nitrite 0.1-1.9%, phosphate 0-5%, sodium ascorbate 0.04-0.07 , glucose 0.03-0.6, spices, water 20-50%.

### EXAMPLE 8

### Pharmaceuticals Anti constipation and Antibiotics

### EXAMPLE 8.1

A composition 0.1-20% yellow mustard gum, water, mixed, homogenized, packaged (bottles, cans, pouches) pasteurized/sterilized.

### EXAMPLE 8.2

A composition containing 0.1-20% yellow mustard gum, citric acid, food coloring materials, flavoring agent, etc commonly used in juices was mixed, packaged (bottles, cans, pouches) sterilized pasteurized and packed for used as laxatives.

### EXAMPLE 8.3

A composition containing 0.1-20% yellow mustard gum, citric acid, tartaric acid, acid, sugars, food coloring materials, flavoring agent, etc commonly used in juices was mixed, packaged (bottles, cans, pouches) pasteurized and packed for used as laxatives.

### EXAMPLE 8.4

A dry mix containing 0.1-20% yellow mustard gum, citric acid, tartaric acid, acid, sugars, food coloring materials, flavoring agent, etc commonly used in juices was mixed, packaged (bottles, cans, pouches, tablets, capsules) used as laxatives.

### EXAMPLE 8.5

A dry mix containing 0.1-20% yellow mustard gum, citric acid, food coloring materials, flavoring agent, etc commonly used in juices was mixed, packaged (bottles, cans, pouches, tablets, capsules) used as laxatives.

### EXAMPLE 8.6

### Pharmaceutical Antibiotic Base Cream

Sterilized yellow mustard gum solutions 1-5% sterilized lanolinl 0-70%, Mix and homogenize the ingredient and package the base cream aseptically. This is used as a base for antibiotic creams by addition of antibiotics, anti-bacterial agents, and anti-fungal agents.

### EXAMPLE 8.7

### Pharmaceutical Antibiotic Base Cream

Sterilized yellow mustard gum solutions 1-5%, sterilized Petrolatum 10-70%, zinc oxide 1-25%. Mix and homogenize the ingredient and package the base cream aseptically. Mix the ingredient, sterilize and package the base cream. This is used as a base for antibiotic cream by addition of antibiotics, anti-bacterial agents, and anti-fungal agents.

### EXAMPLE 8.8

### Antibiotic Creams

Dry powder antibiotics such as penicillin, amoxicillin, neomycin, sterilized lanolin, zinc oxide, and sterilized yellow mustard gum solution were mixed, homogenized aseptically and packaged.

### EXAMPLE 8.9

### Antibiotic Gels

Dry powder antibiotics such as penicillin, amoxicillin, neomycin and zinc oxide are added to the sterilized yellow mustard gum solution mixed, and homogenized aseptically and packaged.

### EXAMPLE 9

### Cosmetics

Varieties of cosmetic products were produced containing yellow mustard gum for use and emulsifying agent to remove the oils and dirt from skin especially facial cleaner with and without soaps.

### EXAMPLE 9.1

### Hair Conditioner

A composition containing yellow mustard gum 0.1-20%, lactic acid, perfumes, natural colors, preservatives, disodium EDTA 0.02-0.1%, water to 100.

### EXAMPLE 9.2

### Shampoo

A composition containing: yellow mustard gum 1-2%, Triethanolamine lauryl sulphate, 25%, coconut diethanolamide 10%, coloring, perfume, preservatives, water to 100%.

### EXAMPLE 9.3

### Toothpaste (Gel)

A composition containing: yellow mustard gum 1%, glycerin 10-30%, sodium lauryl sulfate 0.1-2%, peppermint oil 0.1-2%, xylitol (sweetener) 0.15-20%, abrasive silica 10-50%, coloring agent 0.01-0.5%, Na2FPO3 or sodium fluoride 0.02-1%,

### EXAMPLE 9.4

### Cream Mascara

A composition containing: yellow mustard gum 1-40%, sugar syrup 35%, black ivory (or umber) 25%.

### EXAMPLE 9.5

### Liquid Lipstick

Yellow Mustard Gum 3%, ethyl alcohol 68.5%, petroleum ether, 20%, hydrogenated methyl abeitate 7.5%, rhodamine, 1.%.

### EXAMPLE 9.6

### Liquid Rouge

Yellow mustard Gum 2%, surfactant 0.1-0.2%, water soluble red, preservatives, water.

### EXAMPLE 10

### Emulsion Paints

The followings are two basic emulsion paint formulations using yellow mustard gum as the thickener and rheology modifier. Varieties of paints formulations are possible using yellow mustard gum, in associations with other chemicals used in paints formulations known to the experts in the field. Yellow mustard gum is unique since it has excellent shear thinning properties, excellent emulsifier and excellent suspension ability, which are required in manufacturing of stable paint. The following are three basic formulations for white, yellow and red paints. By changing the pigments, varieties of colors could be produced.

### EXAMPLE 10.1

### Basic White Paint (Vinyl Acetate) Latex

Titanium dioxidel 5%, calcium carbonate 7%, sodium polyphosphate or potassium polyphosphate 2%, butyl carbitol 2.5%, biocide 0.5%, antifoam 0.35, ammonium hydroxide (28) 0.2%, yellow mustard gum 0.5%, vinyl acetate/vinyl versatate latex 43%, and the remainder water.

### EXAMPLE 10.2

### Basic Yellow Paint (Vinyl Acetate) Latex

Titanium dioxidel 0%, 7% yellow iron oxide, calcium carbonate 7%, sodium polyphosphate or potassium polyphosphate 2%, butyl carbitol 2.5%, biocide 0.5%, antifoam 0.35, ammonium hydroxide (28) 0.2%, yellow mustard gum 0.5%, vinyl acetate/vinyl versatate latex 43%, and the remainder water.

### EXAMPLE 10.3

### Basic Red Paint (Vinyl Acetate) Latex

Titanium dioxidel 0%, 10% red iron oxide, calcium carbonate 7%, sodium polyphosphate or potassium polyphosphate 2%, butyl carbitol 2.5%, biocide 0.5%, antifoam 0.35, ammonium hydroxide (28) 0.2%, yellow mustard gum 0.5%, vinyl acetate/vinyl versatate latex 43%, and the remainder water.

### EXAMPLE 10.4

### Basic White Paint (Vinyl Acrylate) Latex

Titanium dioxidel 5%, calcium carbonate 7%, sodium polyphosphate or potassium polyphosphate 2%, butyl carbitol 2.5%, biocide 0.5%, antifoam 0.35, ammonium hydroxide (28) 0.2%, yellow mustard gum 0.5%, vinyl acrylate/acrylic acid latex 43%, and the remainder water.

### EXAMPLE 10.5

### Basic Yellow Paint (Vinyl Acrylate) Latex

Titanium dioxidel 0%, 7% yellow iron oxide, calcium carbonate 7%, sodium polyphosphate or potassium polyphosphate 2%, butyl carbitol 2.5%, biocide 0.5%, antifoam 0.35, ammonium hydroxide (28) 0.2%, yellow mustard gum 0.5%, vinyl acrylate/acrylic acid latex 43%, and the remainder water.

### EXAMPLE 10.6

### Basic Red Paint (Vinyl Acrylate) Latex

titanium dioxidel 0%, 10% red iron oxide , calcium carbonate 7%, sodium polyphosphate or potassium polyphosphate 2%, butyl carbitol 2.5%, biocide 0.5%, antifoam 0.35, ammonium hydroxide (28) 0.2%, yellow mustard gum 0.5%, vinyl acrylate/acrylic acid latex 43%, and the remainder water.

### EXAMPLE 11

### Baking Products

### EXAMPLE 11.1

### Wheat Bread with Yellow Mustard Gum

1-3% yellow mustard gum, wheat flour, salt, sugar, yeast, and baking soda, water. Mix the ingredients, leaven the dough, and bake.

### EXAMPLE 11.2

### Wheat and Rye Bread with Yellow Mustard Gum

1-3% yellow mustard gum, wheat flour, rye flour, salt, sugar, yeast, baking soda, water. Mix the ingredients, leaven the dough and bake.

### EXAMPLE 11.3

### Wheat, Rye, and Oat Bread with Yellow Mustard Gum

1-3% yellow mustard gum, wheat flour, rye flour, Oat flour, salt, sugar, yeast, baking soda, water. Mix the ingredients leaven the dough and bake.

### EXAMPLE 11.4

### Muffins

Yellow Mustard Gum Muffins: 1-3% yellow mustard gum, wheat flour, rye flour, Oat flour, vegetable shortening, salt, sugar, baking soda, milk, raisin, nuts, flavoring, colors. Mix the ingredients, leaven the batter and bake.

### EXAMPLE 11.5

### Biscuits

Yellow Mustard Gum Biscuits: 1-3% yellow mustard gum, wheat flour, rye flour, Oat flour, vegetable shortening, salt, sugar, baking soda, milk, nuts, flavoring, colors. Mix the ingredients, leaven the dough, roll and flatten the dough, cut to pieces and bake.

### EXAMPLE 12

### Solutions of Yellow Mustard Gums

### EXAMPLE 12.1

Yellow Mustard Gum solutions 0.1-1% were prepared by adding 0.1, 0.2, 0.3, 0.4, 0.5, 0.6, 0.7, 0.8, 0.9, and 1 g of mustard gum to water, then mixed and homogenized, packed and pasteurized/sterilized.

### EXAMPLE 12.2

Yellow Mustard Gum solutions 1-35% were prepared by adding 1, 2, 3, 4, 5, 6, 7, 8, 9, 10 . . . 31, 32, 33, 34, 35 g of mustard gum to water, then mixed and homogenized, packed and pasteurized/sterilized.

### EXAMPLE 12.3

The mixtures of yellow mustard gum with galactomanan solutions at 0.1-1% were prepared. Solutions of yellow mustard gum and galactomanan were prepared individually and then mixed to provide a ratio of yellow mustard gum to galactomanan ranging from 1:9 to 9:1.

### EXAMPLE 13

### Dry Mixes

Dry mix of yellow mustard gum and galactomanan were prepared by mixing the dry gums to provide a mixture comprising of yellow mustard gum and galactomanan with ratios ranging from 1:9 to 9:1.

### EXAMPLE 13.1

1 part yellow mustard gum 9 part galactomanan

### EXAMPLE 13.2

2 part yellow mustard gum 8 part galactomanan

### EXAMPLE 13.3

3 part yellow mustard gum 7 part galactomanan

### EXAMPLE 13.4

4 part yellow mustard gum 6 part galactomanan

### EXAMPLE 13.9

5 part yellow mustard gum 5 part galactomanan

### EXAMPLE 13.9

6 part yellow mustard gum 4 part galactomanan

### EXAMPLE 13.9

7 part yellow mustard gum 3 part galactomanan

### EXAMPLE 13.9

8 part yellow mustard gum 2 part galactomanan

### EXAMPLE 13.9

9 part yellow mustard gum 1 part galactomanan

### EXAMPLE 14

### Dry Mixes

Dry mix of yellow mustard gum and Arabic gum were prepared by mixing the dry gums to provide a mixture comprising of yellow mustard gum and Arabic gum with ratios ranging from 1:9 to 9:1.

### EXAMPLE 14.1

1 part yellow mustard gum 9 part Arabic gum

### EXAMPLE 13.2

2 part yellow mustard gum 8 part Arabic gum

### EXAMPLE 14.3

3 part yellow mustard gum 7 part Arabic gum

### EXAMPLE 14.4

4 part yellow mustard gum 6 part Arabic gum

### EXAMPLE 14.5

5 part yellow mustard gum 5 part Arabic gum

### EXAMPLE 14.6

6 part yellow mustard gum 4 part Arabic gum

### EXAMPLE 14.7

7 part yellow mustard gum 3 part Arabic gum

### EXAMPLE 14.8

8 part yellow mustard gum 2 part Arabic gum

### EXAMPLE 14.9

9 part yellow mustard gum 1 part Arabic gum

### EXAMPLE 15

### Dry Mixes

Dry mix of yellow mustard gum and guar gum were prepared by mixing the dry gums to provide a mixture comprising of yellow mustard gum and guar gum with ratios ranging from 1:9 to 9:1

### EXAMPLE 15.1

1 part yellow mustard gum 9 part guar gum

### EXAMPLE 15.2

2 part yellow mustard gum 8 part guar gum

### EXAMPLE 15.3

3 part yellow mustard gum 7 part guar gum

### EXAMPLE 15.4

4 part yellow mustard gum 6 part guar gum

### EXAMPLE 15.5

5 part yellow mustard gum 5 part guar gum

### EXAMPLE 15.6

6 part yellow mustard gum 4 part guar gum

### EXAMPLE 15.7

7 part yellow mustard gum 3 part guar gum

### EXAMPLE 15.8

8 part yellow mustard gum 2 part guar gum

### EXAMPLE 15.9

9 part yellow mustard gum 1 part guar gum

### EXAMPLE 16

### Dry Mixes

Dry mix of yellow mustard gum and locust bean gum were prepared by mixing the dry gums to provide a mixture comprising of yellow mustard gum and locust bean gum with ratios ranging from 1:9 to 9:1

### EXAMPLE 16.1

1 part yellow mustard gum 9 part locust bean gum

### EXAMPLE 16.2

2 part yellow mustard gum 8 part locust bean gum

### EXAMPLE 16.3

3 part yellow mustard gum 7 part locust bean gum

### EXAMPLE 16.4

4 part yellow mustard gum 6 part locust bean gum

### EXAMPLE 16.5

5 part yellow mustard gum 5 part locust bean gum

### EXAMPLE 16.6

6 part yellow mustard gum 4 part locust bean gum

### EXAMPLE 16.7

7 part yellow mustard gum 3 part locust bean gum

### EXAMPLE 16.8

8 part yellow mustard gum 2 part locust bean gum

### EXAMPLE 16.9

9 part yellow mustard gum 1 part locust bean gum

### EXAMPLE 17

### Dry Mix

Dry mix of yellow mustard gum and tragacanth gum were prepared by mixing the dry gums to provide a mixture comprising of yellow mustard gum and tragacanth gum with ratios ranging from 1:9 to 9:1

### EXAMPLE 17.1

1 part yellow mustard gum 9 part tragacanth gum

### EXAMPLE 17.2

2 part yellow mustard gum 8 part tragacanth gum

### EXAMPLE 17.3

3 part yellow mustard gum 7 part tragacanth gum

### EXAMPLE 17.4

4 part yellow mustard gum 6 part tragacanth gum

### EXAMPLE 17.5

5 part yellow mustard gum 5 part tragacanth gum

### EXAMPLE 17.6

6 part yellow mustard gum 4 part tragacanth gum

### EXAMPLE 17.7

7 part yellow mustard gum 3 part tragacanth gum

### EXAMPLE 17.8

8 part yellow mustard gum 2 part tragacanth gum

### EXAMPLE 17.9

9 part yellow mustard gum 1 part tragacanth gum

### EXAMPLE 18

### Dry Mix

Dry mix of yellow mustard gum and CMC gum were prepared by mixing the dry gums to provide a mixture comprising of yellow mustard gum and CMC gum with ratios ranging from 1:9 to 9:1

### EXAMPLE 18.1

1 part yellow mustard gum 9 part CMC gum

### EXAMPLE 18.2

2 part yellow mustard gum 8 part CMC gum

### EXAMPLE 18.3

3 part yellow mustard gum 7 part CMC gum

### EXAMPLE 18.4

4 part yellow mustard gum 6 part CMC gum

### EXAMPLE 18.5

5 part yellow mustard gum 5 part CMC gum

### EXAMPLE 18.6

6 part yellow mustard gum 4 part CMC gum

### EXAMPLE 18.7

7 part yellow mustard gum 3 part CMC gum

### EXAMPLE 18.8

8 part yellow mustard gum 2 part CMC gum

### EXAMPLE 18.9

9 part yellow mustard gum 1 part CMC gum

### EXAMPLE 19

### Dry Mix

Dry mix of yellow mustard gum and cellulose derived gum were prepared by mixing the dry gums to provide a mixture comprising of yellow mustard gum and cellulose derived gum with ratios ranging from 1:9 to 9:1

### EXAMPLE 19.1

1 part yellow mustard gum 9 part cellulose derived gum

### EXAMPLE 19.2

2 part yellow mustard gum 8 part cellulose derived gum

### EXAMPLE 19.3

3 part yellow mustard gum 7 part cellulose derived gum

### EXAMPLE 19.4

4 part yellow mustard gum 6 part cellulose derived gum

### EXAMPLE 19.5

5 part yellow mustard gum 5 part cellulose derived gum

### EXAMPLE 19.6

6 part yellow mustard gum 4 part cellulose derived gum

### EXAMPLE 19.7

7 part yellow mustard gum 3 part cellulose derived gum

### EXAMPLE 19.8

8 part yellow mustard gum 2 part cellulose derived gum

### EXAMPLE 19.9

9 part yellow mustard gum 1 part cellulose derived gum

### EXAMPLE 20

Dry Mix

Dry mix of yellow mustard gum and starch were prepared by mixing the dry gums to provide a mixture comprising of yellow mustard gum and starch with ratios ranging from 1:9 to 9:1

### EXAMPLE 20.1

1 part yellow mustard gum 9 part starch

### EXAMPLE 20.2

2 part yellow mustard gum 8 part starch

### EXAMPLE 20.3

3 part yellow mustard gum 7 part starch

### EXAMPLE 20.4

4 part yellow mustard gum 6 part starch

### EXAMPLE 20.5

5 part yellow mustard gum 5 part starch

### EXAMPLE 20.6

6 part yellow mustard gum 4 part starch

### EXAMPLE 20.7

7 part yellow mustard gum 3 part starch

### EXAMPLE 20.8

8 part yellow mustard gum 2 part starch

### EXAMPLE 20.9

9 part yellow mustard gum 1 part starch

### EXAMPLE 21

### Dry Mix

Dry mix of yellow mustard gum and dextrin were prepared by mixing the dry gums to provide a mixture comprising of yellow mustard gum and dextrin with ratios ranging from 1:9 to 9:1

### EXAMPLE 21.1

1 part yellow mustard gum 9 part dextrin

### EXAMPLE 21.2

2 part yellow mustard gum 8 part dextrin

### EXAMPLE 21.3

3 part yellow mustard gum 7 part dextrin

### EXAMPLE 21.4

4 part yellow mustard gum 6 part dextrin

### EXAMPLE 21.5

5 part yellow mustard gum 5 part dextrin

### EXAMPLE 21.6

6 part yellow mustard gum 4 part dextrin

### EXAMPLE 21.7

7 part yellow mustard gum 3 part dextrin

### EXAMPLE 21.8

8 part yellow mustard gum 2 part dextrin

### EXAMPLE 21.9

9 part yellow mustard gum 1 part dextrin

### EXAMPLE 22

### Dry Mix

Dry mix of yellow mustard gum and alginates were prepared by mixing the dry gums to provide a mixture comprising of yellow mustard gum and alginates, with ratios ranging from 1:9 to 9:1

### EXAMPLE 22.1

1 part yellow mustard gum 9 part alginates

### EXAMPLE 22.2

2 part yellow mustard gum 8 part alginates

### EXAMPLE 22.3

3 part yellow mustard gum 7 part alginates

### EXAMPLE 22.4

4 part yellow mustard gum 6 part alginates

### EXAMPLE 22.5

5 part yellow mustard gum 5 part alginates

### EXAMPLE 22.6

6 part yellow mustard gum 4 part alginates

### EXAMPLE 22.7

7 part yellow mustard gum 3 part alginates

### EXAMPLE 22.8

8 part yellow mustard gum 2 part alginates

### EXAMPLE 22.9

9 part yellow mustard gum 1 part alginates

### EXAMPLE 23

### Dry Mix

Dry mix of yellow mustard gum and agar were prepared by mixing the dry gums to provide a mixture comprising of yellow mustard gum and agar with ratios ranging from 1:9 to 9:1

### EXAMPLE 23.1

1 part yellow mustard gum 9 part agar

### EXAMPLE 23.2

2 part yellow mustard gum 8 part agar

### EXAMPLE 23.3

3 part yellow mustard gum 7 part agar

### EXAMPLE 23.4

4 part yellow mustard gum 6 part agar

### EXAMPLE 23.5

5 part yellow mustard gum 5 part agar

### EXAMPLE 23.6

6 part yellow mustard gum 4 part agar

### EXAMPLE 23.7

7 part yellow mustard gum 3 part agar

### EXAMPLE 23.8

8 part yellow mustard gum 2 part agar

### EXAMPLE 23.9

9 part yellow mustard gum 1 part agar.

### EXAMPLE 24

### Emulsion Systems

Emulsions were made by adding solutions of yellow mustard gums in water at 0.5-2% concentrations to edible and inedible oils 10-90% followed by mixing in a blender. These emulsion system are used in foods as well as a base for pharmaceutical creams.

### EXAMPLE 24.1

Canola oil at concentration of 10-90% was added to solutions of yellow mustard gums in water at 0.5-2% concentrations followed by mixing in a blender, sterilization and packaging.

### EXAMPLE 24.2

10% canola oil, 90% the above yellow mustard gum solutions

### EXAMPLE 24.3

20% canola oil, 80% the above yellow mustard gum solutions

### EXAMPLE 24.4

30% canola oil, 70% the above yellow mustard gum solutions

### EXAMPLE 24.5

40% canola oil, 60% the above yellow mustard gum solutions

### EXAMPLE 24.6

50% canola oil, 50% the above yellow mustard gum solutions

### EXAMPLE 24.7

60% canola oil, 40% the above yellow mustard gum solutions

### EXAMPLE 24.8

70% canola oil, 30% the above yellow mustard gum solutions

### EXAMPLE 24.9

80% canola oil, 20% the above yellow mustard gum solutions

### EXAMPLE 24.10

90% canola oil, 10% the above yellow mustard gum solutions

### EXAMPLE 24.11

Olive oil at concentration of 10-90% was added to solutions of yellow mustard gums in water at 0.5-2% concentrations followed by mixing in a blender, sterilization and packaging

### EXAMPLE 24.12

10% olive oil, 90% the above yellow mustard gum solutions

### EXAMPLE 24.13

20% olive oil, 80% the above yellow mustard gum solutions

### EXAMPLE 24.14

30% olive oil, 70% the above yellow mustard gum solutions

### EXAMPLE 24.15

40% olive oil, 60% the above yellow mustard gum solutions

### EXAMPLE 24.16

50% olive oil, 50% the above yellow mustard gum solutions

### EXAMPLE 24.17

60% olive oil, 40% the above yellow mustard gum solutions

### EXAMPLE 24.18

70% olive oil, 30% the above yellow mustard gum solutions

### EXAMPLE 24.19

80% olive oil, 20% the above yellow mustard gum solutions

### EXAMPLE 24.20

90% olive oil, 10% the above yellow mustard gum solutions.

### EXAMPLE 24.21

Cooking oil at concentration of 10-90% was added to solutions of yellow mustard gums in water at 0.5-2% concentrations followed by mixing in a blender, sterilization and packaging

### EXAMPLE 24.22

10% cooking oil, 90% the above yellow mustard gum solutions

### EXAMPLE 24.23

20% cooking oil, 80% the above yellow mustard gum solutions

### EXAMPLE 24.24

30% cooking oil, 70% the above yellow mustard gum solutions

### EXAMPLE 24.25

40% cooking oil, 60% the above yellow mustard gum solutions

### EXAMPLE 24.26

50% cooking oil, 50% the above yellow mustard gum solutions

### EXAMPLE 24.27

60% cooking oil, 40% the above yellow mustard gum solutions

### EXAMPLE 24.28

70% cooking oil, 30% the above yellow mustard gum solutions

### EXAMPLE 24.29

80% cooking oil, 20% the above yellow mustard gum solutions

### EXAMPLE 24.30

90% cooking oil,10% the above yellow mustard gum solutions

### EXAMPLE 24.31

Mineral oil at concentration of 10-90% was added to solutions of yellow mustard gums in water at 0.5-2% concentrations followed by mixing in a blender, sterilization and packaging

### EXAMPLE 24.32

10% mineral oil, 90% the above yellow mustard gum solutions

### EXAMPLE 24.33

20% mineral oil, 80% the above yellow mustard gum solutions

### EXAMPLE 24.34

30% mineral oil, 70% the above yellow mustard gum solutions

### EXAMPLE 24.35

40% mineral oil, 60% the above yellow mustard gum solutions

### EXAMPLE 24.36

50% mineral oil, 50% the above yellow mustard gum solutions

### EXAMPLE 24.37

60% mineral oil, 40% the above yellow mustard gum solutions

### EXAMPLE 24.38

70% mineral oil, 30% the above yellow mustard gum solutions

### EXAMPLE 24.39

80% mineral oil, 20% the above yellow mustard gum solutions

### EXAMPLE 24.40

90% mineral oil, 10% the above yellow mustard gum solutions

### EXAMPLE 24.41

Petrolatum at concentration of 10-90% was added to solutions of yellow mustard gums in water at 0.5-2% concentrations followed by mixing in a blender, sterilization and packaging

### EXAMPLE 24.42

10% Petrolatum, 90% the above yellow mustard gum solutions

### EXAMPLE 24.43

20% Petrolatum, 80% the above yellow mustard gum solutions

### EXAMPLE 24.44

30% Petrolatum, 70% the above yellow mustard gum solutions

### EXAMPLE 24.45

40% Petrolatum, 60% the above yellow mustard gum solutions

### EXAMPLE 24.46

50% Petrolatum, 50% the above yellow mustard gum solutions

### EXAMPLE 24.47

60% Petrolatum, 40% the above yellow mustard gum solutions

### EXAMPLE 24.48

70% Petrolatum, 30% the above yellow mustard gum solutions

### EXAMPLE 24.49

80% Petrolatum, 20% the above yellow mustard gum solutions

### EXAMPLE 24.50

90% Petrolatum, 10% the above yellow mustard gum solutions

### EXAMPLE 24.51

Cotton seed oil at concentration of 10-90% was added to solutions of yellow mustard gums in water at 0.5-2% concentrations followed by mixing in a blender, sterilization and packaging

### EXAMPLE 24.52

10% cotton seed oil, 90% the above yellow mustard gum solutions

### EXAMPLE 24.53

20% cotton seed oil, 80% the above yellow mustard gum solutions

### EXAMPLE 24.54

30% cotton seed oil, 70% the above yellow mustard gum solutions

### EXAMPLE 24.55

40% cotton seed oil, 60% the above yellow mustard gum solutions

### EXAMPLE 24.56

50% cotton seed oil, 50% the above yellow mustard gum solutions

### EXAMPLE 24.57

60% cotton seed oil, 40% the above yellow mustard gum solutions

### EXAMPLE 24.58

70% cotton seed oil, 30% the above yellow mustard gum solutions

### EXAMPLE 24.50

80% cotton seed oil, 20% the above yellow mustard gum solutions

### EXAMPLE 24.60

90% cotton seed oil, 10% the above yellow mustard gum solutions

### EXAMPLE 24.61

Motor oil at concentration of 10-90% was added to solutions of yellow mustard gums in water at 0.5-2% concentrations followed by mixing in a blender.

### EXAMPLE 24.62

10% motor oil, 90% the above yellow mustard gum solutions

### EXAMPLE 24.63

20% motor oil, 80% the above yellow mustard gum solutions

### EXAMPLE 24.64

30% motor oil, 70% the above yellow mustard gum solutions

### EXAMPLE 24.65

40% motor oil, 60% the above yellow mustard gum solutions

### EXAMPLE 24.66

50% motor oil, 50% the above yellow mustard gum solutions

### EXAMPLE 24.67

60% motor oil, 40% the above yellow mustard gum solutions

### EXAMPLE 24.68

70% motor oil, 30% the above yellow mustard gum solutions

### EXAMPLE 24.69

80% motor oil, 20% the above yellow mustard gum solutions

### EXAMPLE 24.70

90% motor oil, 10% the above yellow mustard gum solutions

1. A composition comprising about 0.1 to 35% yellow mustard gum by weight wherein the balance of said composition is selected from the group consisting of: water, oil in water emulsion, water in oil emulsion, beverage, juice, sauce, milk, milk derivatives, wine, liquors, alcohol, spirit, esters, soluble and insoluble, edible and inedible compounds, a gel of water, oil and solids, a paste of water, oil and solids, a dry mix of other solid compounds.

2. The process of claim 1 wherein
A. (a-g) comprising soaking or dissolving yellow mustard gum in liquid for a period of some times followed by mixing and homogenization, pH adjustment acidic<7 basic>7 and neutral=7, followed by additions of other edible and inedible solids and liquid substances, wherein the said edible substances are oils, solids, soluble materials comprising sugars, sweetners, salts, acids (citric acid, acetic acid, ascorbic acid, tartaric acid), milk powder, whey, powder, dried dairy powder, dried fruits, dried vegetables, spices, mustard seeds including yellow mustard seeds, vegetable proteins, meats, fats, oils, lecithin, galactomannan, phenolics, esters, food additives, food preservatives, food coloring, followed by mixing whereby said, emulsion, gel and paste is formed and stabilized and the finished products are packaged followed by pasteurization and or sterilization. And the said inedible substances are the chemicals used in paints, cosmetics, pharmaceutical and chemistry industries listed in the list of definition.
B. (h) comprising of mixing dried yellow mustard gum with one or more of the following edible and inedible dry solids wherein, the said edible solids comprising of sugars, sweetners, salts, acids (citric acid, acetic acid, ascorbic acid, tartaric acid), milk powder, whey, powder, dried dairy powder, dried fruits, dried vegetables, spices, mustard seeds including yellow mustard seeds, vegetable proteins, meats, fats, oils, lecithin, galactomannan, phenolics, esters, food additives, food preservatives, food coloring, industrial gums such as guar gum, Arabic gum, CMC, xanthan gum, agar, tragacaunth, galactomannan and medical compounds followed by mixing and packaging of the said resultant dry mix in bottles, jars, bags, capsules tablets, etc. and that the said inedible solids and substances are the chemicals used in paints, cosmetics, pharmaceutical and chemical industries listed in the list of definition.

3. A composition of claim 1 in the form of liquids, pastes, gels, creams, dry-mixes, bulked or packaged in any form in jars, cans, glasses, tablets, capsules, or other forms, and wherein, the said composition is pasteurized and sterilized by conventional heating, microwave heating including RF frequencies, UV pasteurization, HTST, batch and continuous sterilization and pasteurization, and flash sterilization, and irradiation.

4. A composition of claim 1 selected from the group consisting of edible and inedible gel, emulsions, and paste products comprising nutraceutical, pharmaceutical, cosmetics, paints and chemical derivative products, such as nutraceutical beverages, nutraceutical confectionary, nutraceutical baking products, nutraceutical meat products, nutraceutical salad dressings, nutraceutical liquid sauces, nutraceutical paste sauces, nutraceutical dairy products, nutraceutical medical compounds and inedible products comprising medicines, cosmetics, paints and other chemicals, and the said products are packaged in glass, cans, jars, pouches, capsules, tablets (coated and uncoated) are stabilized, pasteurized, and sterilized.

5. A composition of claim 1, comprising a liquid sauce selected from the group consisting of soy sauce, teriyaki sauce, yakitori sauce, ponzu sauce, tsukiyaki sauce, sukiyaki sauce, szechuan sauce, mango sauce, tomato sauce, pizza sauce, barbeque sauce, and chili sauce.

6. Process of claim (1c.) wherein, said nutraceutical beverage is a plain and or carbonated beverage comprising of yellow mustard gum having a concentration ranging from about 0.1-1% in combination with at least one, two or more juice from the following fruit and or vegetable juice concentrates: apple juice concentrate, apricot juice concentrate, banana juice concentrate, barberry juice concentrates, blueberry juice concentrate, black berry juice concentrates, cantaloupe juice concentrate, cherry juice concentrate, cranberry juice concentrate, rose sherbet, grape juice concentrate, grapefruit juice concentrate, guava juice concentrates, guava puree, kale juice concentrate, kiwi fruit juice concentrate, lemon and lime juice concentrates, lychee juice concentrate, mandarins juice concentrate, mango juice concentrate, mango puree, mangosteen juice concentrates, melon juice concentrates, nashi juice concentrates, nectarines juice concentrate, orange juice concentrate, papaya juice concentrate, passion fruit juice concentrate, peach juice concentrates, peach puree, pear juice concentrate, pear puree, parmesan juice concentrate, pineapple juice concentrate, pomegranate juice concentrate, pomegranate puree, pomelo juice concentrate, prune juice concentrate, raspberry juice concentrate, rose water sherbet, strawberry juice concentrate, star fruit juice concentrate, tangerine juice, tamarillo juice concentrate, water melon juice concentrates, carrot juice concentrate, celery juice concentrate, green barley juice concentrate, green lettuce juice concentrate, parsley juice concentrate, rhubarb juice concentrate, spinach juice concentrate, thyme juice concentrates, and tomato juice concentrate, etc.

7. Process of claim 1, wherein the said solids, soluble materials, liquids comprising edible and inedible materials such as: fruits, vegetables, vegetable protein, lecithin, fresh or processed meats, seeds, including mustard seeds and yellow mustard seeds, spices, milk powder, whey powder, and dried/dry dairy products, oils comprising vegetable or animal fats such as canola oil, sunflower oil, corn oil, peanut oil, sesame oil, flask oil, rapeseed oil, mustard oil, almond oil, phospholipids, milk butter, milk cream, fish oils, lard, Custer oil, mineral oil, petrochemical derived oils, sugars, sweetners, salts, acids (citric acids, ascorbic acid, tartaric acid, acetic acid, phenolics, esters, FDA approved additives, food coloring, food flavorings, commonly found in fruit juices and commonly used in food and pharmaceuticals and said edible and none-edible soluble materials comprising ionic surfactant, cationic surfactant, nonionic surfactant, pigments, extenders, dispersant, biocide, coalescing agent, film formers, and pH controller, foam boosters, conditioning agent, special additives and preservatives, sequestering agent, viscosity modifiers, clarifying agent, fragrances, colors, and said dry edible and inedible solids comprising of organic inorganic salts, organic inorganic acids, phenolics, esters, additives, pigments, coloring, perfumes, preservatives ionic surfactant, cationic surfactant, nonionic surfactant, pigments, extenders, dispersant, biocide, coalescing agent, film formers, and pH controller, foam boosters, conditioning agent, special additives and preservatives, sequestering agent, viscosity modifiers, clarifying agent, fragrances, colors commonly used in food and pharmaceutical, paints, and other chemical industries.

8. Process of claim (1g) wherein said paste comprising of meat emulsion containing one or more meat from the following meat: beef, mutton, pork, lamb, goat, camel, horse, donkey, dear, turkey, chicken, pheasant, fish, and other meats of wild animals and the said paste (emulsion) is processed at a temperature greater than room temperature up to 250F or alternatively processed by smoke in smoke house preferably up to about 180F, or processed by microwave, or radio frequency heat generators.

9. A nutraceutical composition of claim 4, comprising from about 0.1 to 35% by weight of yellow mustard gum selected from the group consisting of diabetic beverages, diabetic beverages concentrate, prepared food glaze, dry mix diabetic beverages, dry mix food glaze, soy sauce paste, musteriyaki sauce paste, teriyaki sauce paste, yakitori sauce paste, ponzu sauce paste, tsukiyaki sauce paste, sukiyaki sauce paste, szechuan sauce paste, mango sauce paste, tomato sauce paste, pizza sauce paste, barbeque sauce paste, chili sauce paste, mayonnaise, French salad dressing, Italian salad dressing, ranch salad dressing, low fat salad dressing, no fat salad dressing, and their dry premixes, dairy emulsions such as ice cream, milk shake, yogurt, sour creams, cheese, plain milk shake, coffee milkshake, chocolate milkshake, coca milkshake, vanilla milkshake, sour milkshake, sour milkshake with fruits, flavored sour milkshake, plain yogurt, flavored yogurt, plain ice cream, fruit ice cream, cheese, food gels, meat and vegetable gels, sausages, salami, pepperoni, hotdogs, vegetable sausages, canned meat products, confectionary and baking products, bread, cakes, muffins, cookies, and biscuits, cosmetics, gels, shampoo, hair conditioners, mascara, toothpaste, liquid soaps, lipsticks, pharmaceutical gels, body oil remover, moisturizing cream, shaving cream, cough syrups, medical syrups, cream bases, gel bases, antibiotic and antiseptic creams and gels, vinyl acrylic latex paints, vinyl acetate paints, acrylic latex paints.

10. Process of claim 4, wherein, the said meat is a gel meat product formed by the yellow mustard gum during the processing which holds and binds the free water of the meat and its constituents together resulting in a solid dry meat products, and the said gel meat and or meat emulsion comprising of at least one, two and more meat from the list of the following meats: beef, mutton, pork, lamb, goat, camel, horse, donkey, dear, turkey, chicken, pheasant, fish, and other meats of wild animals.

11. Process of claim 4, wherein the said gel comprising of yellow mustard seed gum in combination with galactomannan, and or any other gums and hydrocolloids listed in the definition with a mixing ratio of 1-9 or 9-1 and or 0.1-0.9 or 0.9-0.1.

12. A composition of claim 4, wherein the said composition is pasteurized and sterilized by conventional heating, microwave heating including RF frequencies, UV pasteurization, HTST, batch and continuous sterilization and pasteurization, and flash sterilization, and irradiation.

13. A composition of claim 1, wherein the said emulsion is selected from the group consisting of a food emulsion, a pharmaceutical emulsion, a cosmetic emulsion, a paint emulsion, and a chemical emulsion.

14. Process of claim 4, wherein the said milk is a milk of cow, mare, camel, swine, goat, sheep, donkey, human, dear, moose, and the milk of other wild animals.

15. Process of claim 4, wherein the said dairy products comprising of yellow mustard gum in combination with galactomannan, or other industrial gums such as gum Arabic, guar gum, locust bean gum, tragacanth gum, CMC, cellulose derived gums, starch, dextrin, alginates, agar, whereby the ratio of yellow mustard gum to galactomannan or other gums comprising of 1:9 to 9:1 and or 1% to 99% or 99% to 1%.

16. Process of claim 4, wherein the said solid in (g) comprising of yellow mustard gum in combination with gum Arabic, guar gum, locust bean gum, tragacanth gum, CMC, cellulose derived gums, starch, dextrin, alginates, agar, etc. whereby the ratio of yellow mustard gum to the said gums comprising of 1:9 to 9:1 and or 1% to 99% or 99% to 1%.

17. The composition of claim 4, wherein the said composition comprises yellow mustard gum together with other industrial gums selected from the group consisting of gum Arabic, guar gum, locust bean gum, tragacanth gum, CMC, cellulose derived gums, starch, dextrin, alginates, agar, whereby the ratio of yellow mustard gum to the said gums comprising of 1:9 to 9:1 and or 1% to 99% or 99% to 1%.

18. A composition of claim 4, selected from the group consisting of pharmaceutical gel or cream or emulsion comprising of at least one antibiotic, two antibiotics or more and anti bacterial agent and anti fungal agents such as penicillin, amoxicillin, neomycin, or other antibiotics and drugs listed in the FDA approved list of drugs, etc.

19. Oriental sauce paste comprising yellow mustard gum which on applying on the prepared foods such as hotdogs and sausages stay on the food without slipping selected from the group consisting of soy sauce paste, musteriyaki sauce paste, teriyaki sauce paste, yakitori sauce paste, ponzu sauce paste, tsukiyaki sauce paste, sukiyaki sauce paste, szechuan sauce paste, mango sauce paste, tomato sauce paste, pizza sauce paste, barbeque sauce paste, and chili sauce paste.

20. A salad dressing comprising yellow mustard gum and at least one food acid selected from the group consisting of citric acid, acetic acid, vinegar, tartaric acids and their salts.
